(19) United States
(12) Patent Application Publication
(10) Pub. No.: US 2023/0016478 A1
(43) Pub. Date: Jan. 19, 2023

(54) PLATELET CONCENTRATE CONTROL

(71) Applicant: CYTEGUARD AB, Sollentuna (SE)

(72) Inventor: Ralf GÜTLEIN, Stockstadt (DE)

(21) Appl. No.: 17/785,455

(22) PCT Filed: Dec. 1, 2020
(86) PCT No.: PCT/SE2020/051155

§ 371 Date: Jun. 15, 2022

(30) Foreign Application Priority Data
Dec. 18, 2019 (SE) 1951492-6

(51) Int. Cl. G01N 33/49; G01N 21/51; G01N 21/53

(52) U.S. Cl. CPC G01N 33/49; G01N 21/51; G01N 21/53; G01N 2021/4704

(57) ABSTRACT

An apparatus (1) for determining quality of a platelet concentrate (PC) (15) in a PC bag (10) comprises a movable bag holder (2) to carry the PC bag (10), a light system (20) with a light source (21, 24) to direct light (22) into the platelet concentrate (15) in the PC bag (10) for a measurement interval, and a detector system (30) with a light detector (31, 32) configured to detect light (23) from the platelet concentrate 15 during the measurement interval and generate a real-time detection signal. The apparatus (1) also comprises a controller (40) configured to determine platelet swirling based on the real-time detection signal and determine a quality parameter for the platelet concentrate (15) in the PC bag (10) based on the platelet swirling.

### TECHNICAL FIELD

The present invention generally relates to platelet concentrate control, and in particular to an apparatus and method for determining a quality of a platelet concentrate.

### BACKGROUND

Platelet concentrate (PC), also referred to as thrombocyte concentrate in the art, is used in platelet transfusion to prevent or treat bleeding in patients with low platelet count and/or poor platelet function. Typical patients receiving such platelet transfusions are patients treated with chemotherapy.

Platelet concentrates can be produced either from whole blood or by apheresis and have a shelf life of five to seven days. Storage conditions for platelets differ significantly with regard to temperature as compared to other blood components, such as red blood cells and blood plasma. Today, platelet concentrates are stored in oxygen permeable bags at a temperature of 20-22° C. in dedicated cabinets with temperature control and movable shelves for agitating the platelets. Such agitation is used to prevent aggregation of the platelets and to optimize oxygenation of the platelets in the bags.

Requirements for quality control and documentation are rigorous in the production of blood components. Today, the quality of platelet concentrates is determined by subjective visual inspection of so-called platelet swirling. Platelet swirling is a term for the optical phenomenon that occurs when viable platelets in the bag reflect light in a characteristic manner. Platelets are discoid-shaped and colorless with a unique ability to reflect light. When platelets age and eventually die, they turn round and the property of reflecting light disappears. The swirling effect therefore declines with aging platelet death.

There is currently no objective way to measure platelet swirling in absolute terms. In clear contrast, results of a platelet swirling test are recorded as positive or extensive swirl, moderate or intermediate swirl, and absent or negative swirl. Hence, quality control of platelet concentrates is to date a subjective assessment of the blood bank staff, manufacturing and supplying the platelet concentrate product.

WO 90/14588 discloses an apparatus for continuous quality control of a platelet concentrate. Light sources are arranged to transilluminate bags with platelet concentrates and photodiodes are arranged to detect the light having passed through the bags with platelet concentrates. The detection signal is used to monitor any change in the turbidity or transparency, which is said to reflect the quantity and quality of the platelets in the bags.

U.S. Pat. No. 6,288,778 discloses an apparatus for handling blood in bags. The apparatus comprises a plurality of discs arranged one above another and rotatable about a common central axis. Each disc forms an angle of inclination to the central axis. A light source is arranged beneath each of the discs and a light sensitive member is arranged above each of the discs. Light transmitted by the light sources passes through an aperture in the disc and through the bags to be detected by the light sensitive member. The detected light can be used to measure glare of thrombocytes in blood in a bag retained on the disc.

There is still a need for a technique to determine the quality of platelet concentrates, and in particular such a technique that can detect contaminations in the platelet concentrates.

### SUMMARY

It is a general objective to determine the quality of platelet concentrates.

This and other objectives are met by embodiments as disclosed herein.

The present invention is defined in the independent claims. Further embodiments of the invention are defined in the dependent claims.

An embodiment of the invention relates to an apparatus for determining quality of a platelet concentrate (PC) in a PC bag. The apparatus comprises a movable bag holder configured to carry the PC bag and agitate platelets in the PC bag. The apparatus also comprises a light system comprising at least one light source configured to direct light into the platelet concentrate in the PC bag carried by the movable bag holder for a measurement interval. The apparatus further comprises a detector system comprising at least one light detector configured to detect light from the platelet concentrate in the PC bag during the measurement interval and generate a real-time detection signal. The apparatus additionally comprises a controller connected to the detector system and configured to determine platelet swirling for the platelet concentrate in the PC bag based on the real-time detection signal and determine a quality parameter for the platelet concentrate in the PC bag based on the platelet swirling.

Another embodiment of the invention relates to a method of determining quality of a platelet concentrate in a PC bag. The method comprises directing, for a measurement interval, light into the platelet concentrate in the PC bag carried by a movable bag holder configured to carry the PC bag and agitate platelets in the PC bag. The method also comprises detecting light from the platelet concentrate in the PC bag during the measurement interval. The method further comprises generating a real-time detection signal based on the detected light and determining platelet swirling for the platelet concentrate in the PC bag based on the real-time detection signal. The method additionally comprises determining a quality parameter for the platelet concentrate in the PC bag based on the platelet swirling.

The present invention enables accurately monitoring the quality of platelet concentrates even in cases with contamination in the PC bags. Embodiment of the present invention can also be used to detect any contamination in the platelet concentrates in addition to monitoring the quality of platelets.

### BRIEF DESCRIPTION OF THE DRAWINGS

The embodiments, together with further objects and advantages thereof, may best be understood by making reference to the following description taken together with the accompanying drawings, in which:

FIGS. 1A and 1B illustrate an apparatus for determining quality of a platelet concentrate according to an embodiment;

FIGS. 2A and 2B illustrate an apparatus for determining quality of a platelet concentrate according to another embodiment;

FIG. 3 illustrates an apparatus for determining quality of a platelet concentrate according to a further embodiment;

FIG. 4 illustrates an apparatus for determining quality of a platelet concentrate according to yet another embodiment;

FIG. 5 illustrates an apparatus for determining quality of a platelet concentrate according to yet an embodiment;

FIGS. 6A and 6B illustrate an apparatus for determining quality of a platelet concentrate according to another embodiment;

FIGS. 7A to 7C illustrate an apparatus for determining quality of a platelet concentrate according to a further embodiment;

FIG. 8 illustrates an apparatus for determining quality of a platelet concentrate according to yet another embodiment;

FIG. 9 is a diagram illustrating real-time reflection signal showing swirling platelets determined for a platelet concentrate 2 days and 12 days from production. Amplitude and average are shown;

FIGS. 10A and 10B are diagrams illustrating reflection signal (FIG. 10A) and transmission signal (FIG. 10B) determined for a platelet concentrate lacking microbial contamination represented as average value over time (˜8 days) (RM, TM) and amplitude over time (RS, TS);

FIGS. 11A and 11B are diagrams illustrating reflection signal (FIG. 11A) and transmission signal (FIG. 11B) determined for a platelet concentrate with Staphylococcus epidermidis contamination represented as average value over time (˜8 days) (RM, TM) and amplitude over time (RS, TS);

FIG. 12 is a diagram showing reflectance signal (swirling) and transmission signal average (transmission mean) for non-contaminated platelet concentrate (non-spiked) and contaminated platelet concentrate (spiked);

FIGS. 13A and 13B are diagrams illustrating reflection signal (RS) and transmission signal average (TM) determined for a non-contaminated platelet concentration (FIG. 13A) and a contaminated platelet concentration (FIG. 13B);

FIGS. 14A and 14B are diagrams comparing reflection signal (RS) with visual assessment of swirling (SW) and aggregation at 16 μM collagen (F1), at 8 μM collagen (F2), at 0.5 mg/ml arachidonic acid (ARA) (F5) and at 20 μM thrombin receptor activating peptide (TRAP) (F6) for a non-contaminated platelet concentration (FIG. 14A) and a contaminated platelet concentration (FIG. 14B);

FIG. 15 illustrates an apparatus for determining quality of a platelet concentrate according to an embodiment;

FIG. 16 illustrates an apparatus for determining quality of a platelet concentrate according to another embodiment;

FIG. 17 is a flow chart illustrating a method of determining quality of a platelet concentrate in a PC bag,

FIG. 18 is a flow chart illustrating an additional, optional step of the method shown in FIG. 17 according to an embodiment; and

FIG. 19 is a flow chart illustrating an additional, optional step of the method shown in FIG. 17 according to another embodiment.

### DETAILED DESCRIPTION

Throughout the drawings, the same reference numbers are used for similar or corresponding elements.

The present invention generally relates to platelet concentrate control, and in particular to an apparatus and method for determining a quality of a platelet concentrate.

The prior art apparatuses proposed for continuous quality control of a platelet concentrate uses average transmitted light, i.e., light having passed through the platelet concentrate (PC), in order to monitor any change in turbidity or transparency and using such a turbidity or transparency to monitor the quantity and quality of platelets in the PC bags.

However, experimental data as represented herein indicates that such average transmission signals run into problems in cases of contamination in the platelet concentration, such as by bacteria or other microorganisms. Such bacterial contaminations are relatively more common with platelets as compared to other blood components as they are stored at warmer temperatures.

Any deterioration in the quality of the platelet in the PC bags is, when measuring average light transmission, masked by the contamination since microorganism growth results in a change in transmission that is opposite to the change in average transmission caused by platelet deterioration. As a consequence, an operator or user monitoring the average transmission signal over time may presume that the quality of the platelet concentration is good although a significant portion of the platelets have in deed deteriorated and died if there is microorganism contamination in the monitored PC bag.

The present invention is based on the finding that platelet swirling as determined based on a real-time detection signal is a more appropriate tool for monitoring the quality of platelet concentrates as compared to average transmission signal and other turbidity or transparency representing signals. Any change in platelet quality in the platelet concentrate is accurately represented by the platelet swirling determined based on the real-time detection signal even if the platelet concentrate would be contaminated by microorganisms.

An embodiment of the invention relates to an apparatus for determining quality of a platelet concentrate (PC) in a PC bag. The apparatus comprises a movable bag holder configured to carry the PC bag and agitate platelets in the PC bag. The apparatus also comprises a light system comprising at least one light source configured to direct light into the platelet concentrate in the PC bag carried by the movable bag holder for a measurement interval. The apparatus further comprises a detector system comprising at least one light detector configured to detect light from the platelet concentrate in the PC bag during the measurement interval and generate a real-time detection signal. The apparatus additionally comprises a controller connected to the detector system and configured to determine platelet swirling for the platelet concentrate in the PC bag based on the real-time detection signal and determine a quality parameter for the platelet concentrate in the PC bag based on the platelet swirling.

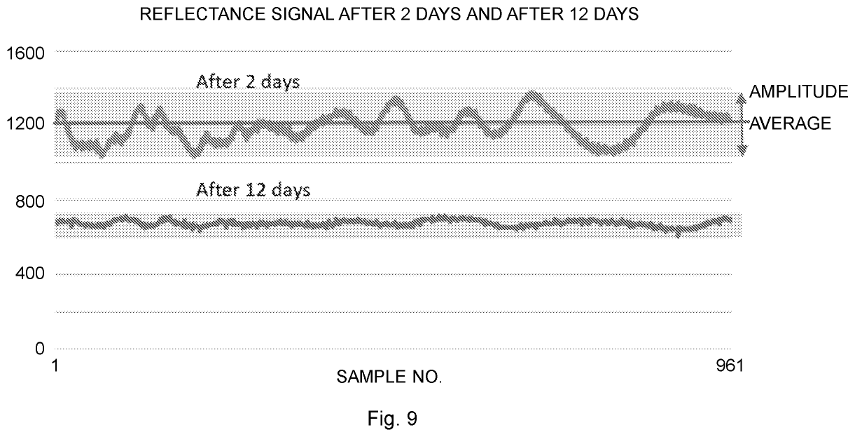

The present information uses a real-time detection signal that reflects detect light during a period of time corresponding to the measurement interval to determine platelet swirling. Such a real-time detection signal accurately reflects the swirling effects caused by platelets in the platelet concentrate in the PC bag. FIG. 9 shows a real-time reflectance signal measured for a platelet concentrate 2 days after filling the PC bag with the platelet concentrate and once more measured after 12 days. After 2 days, most platelets are viable giving rise to a significant swirling effect as can be seen as a large variability or variance in the real-time reflectance signal, sometimes also referred to as time-pendent signal strength of the real-time reflectance signal. However, after 12 days the viability of the platelets is much lower, thereby not producing any significant swirling effect. Accordingly, the variability in the real-time reflectance signal is significantly lower.

Hence, in an embodiment, the controller of the apparatus is configured to determine platelet swirling for the platelet concentrate in the PC bag based on variability or variance of the real-time detection signal and determine the quality parameter for the platelet concentrate in the PC bag based on the platelet swirling.

Variability or variance of the real-time detection signal can be defined in accordance with various embodiments. In an embodiment, the real-time detection signal comprises a plurality of signal samples. The number of such signal samples depend on the sampling frequency of the at least one light detector and the duration of the measurement interval. Each signal sample has a respective value, also denoted sample value herein. In an example, variability or variance could be defined as the, preferably time-dependent, changes in differences or distances between signal values of signal samples and the average of the real-time detection signal, i.e., the average of the signal samples. As is shown in the upper part FIG. 9, there is a large change in sample values relative to the average, i.e., large difference or distance between signal values and the average of the signal values, whereas there is correspondingly much less variation in the signal values relative to the average in the lower part of FIG. 9.

Hence, in an embodiment, the real-time detection signal comprises a plurality of signal samples having a respective sample value. The controller is then configured to calculate an average of the sample values of the signal samples and determine the platelet swirling based on the calculated average and the sample values of the signal samples.

For instance, assume that the real-time detection signal comprises N signal samples for the measurement interval and that sᵢ represents a sample value at sample number i, i=1 . . . N, then the average of the sample values and thereby of the real-time detection signal can be calculated by the controller as

$$\overline{s} = \frac{1}{N} {\sum }_{i = 1}^{N} {s}_{i} .$$

In this embodiment, the controller is, thus, configured to determine the platelet swirling based on the average of the sample values and the sample values of the signal samples, i.e., based on ƒ(s̅, sᵢ) for some function ƒ( ).

In an embodiment, the controller is configured to calculate, for each signal sample of the real-time detection signal, a difference between the sample value of the signal sample and the calculated average. The controller is, in this embodiment, also configured to determine the platelet swirling based on the calculated differences.

In this embodiment, the controller calculates differences or distances Δsᵢ between sample values sᵢ of signal samples and the calculated average s̅ and then determines platelet swirling based on these differences, such as g(Δsᵢ) for some function g( ). Such differences or distances could, for instance, be calculated as Δsᵢ=(sᵢ−s̅) for those sample values sᵢ≥s̅ and Δsᵢ=(s̅−sᵢ) for those sample values sᵢ<s̅, or the controller could calculate absolute differences or squared differences, e.g., Δsᵢ=|sᵢ−s̅| or Δsᵢ=(sᵢ−s̅| or Δsᵢ=(sᵢ−s̅)².

In an embodiment, the controller is configured to determine the platelet swirling based on a sum of the calculated differences. Hence, in this embodiment, the controller is configured to determine platelet swirling based on Σᵢ₌₁^{N}Δsᵢ. Thus, currently preferred examples of determining platelet swirling comprises Σᵢ₌₁^{N}|sᵢ−s̅| and Σᵢ₌₁^{N}(sᵢ−s̅)².

The real-time detection signal in the upper part of FIG. 9 will result in a comparatively larger value of the sum of the calculated differences as compared to the real-time detection signal in the lower part of FIG. 9.

The controller could alternative calculate other parameters representing the variability of the real-time detection signal, preferably relative to the average of the real-time detection signal. For instance, the controller could calculate the area under curve, such as relative to the average of the real-time detection signal. Other parameters include summing the differences or distances Δsᵢ for those signal samples having sample values that are larger than the average of the real-time detection signal to get a first sum and summing the differences or distances Δsᵢ for those signal samples having sample values that are smaller than the average of the real-time detection signal to get a second sum. The controller may then calculate add these two sums, calculate the difference between the absolute value of the first sum and the absolute value of the second sum, calculate the sum of the absolute value of the first sum and the absolute value of the second sum or calculate the quotient between the first sum and the second sum as parameter representing platelet swirling.

In a preferred embodiment, the real-time detection signal is a real-time reflectance signal, i.e., representative of light reflected from the platelet concentration during the measurement interval. In such an embodiment, the detector system comprises a light detector configured to detect reflected light from the platelet concentrate in the PC bag during the measurement interval and generate a real-time reflectance signal representative of light reflected from the platelet concentrate during the measurement interval. In this embodiment, the controller is configured to determine the platelet swirling based on the real-time reflectance signal.

The apparatus of the present invention will now be described with reference to various embodiments illustrated in the drawings.

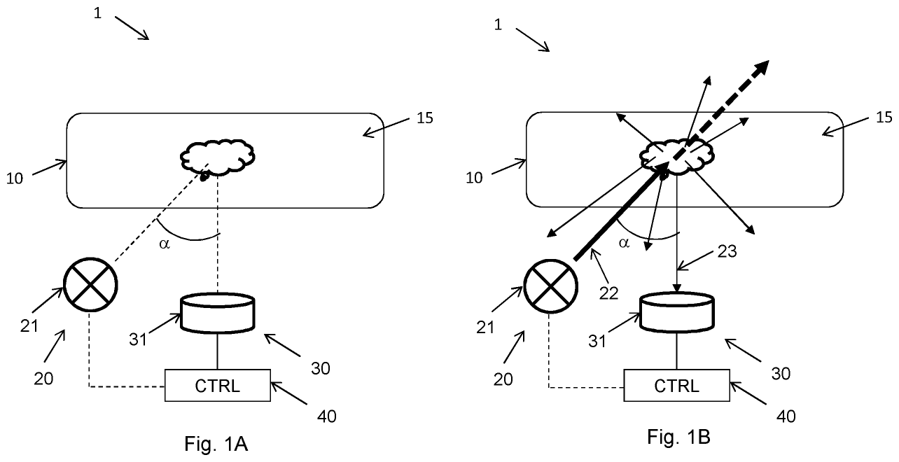

FIGS. 1A and 1B illustrate an embodiment of the apparatus 1 for determining quality of a platelet concentrate 15 in a PC bag 10. In this embodiment, the light system 20 comprises a light source 21 arranged to direct light 22 into the platelet concentrate 15 in the PC bag 10 at an angle of incidence α selected within an interval of from 5° to 85° for the measurement interval. The detector system 30 comprises, in this embodiment, a light detector 31 configured to detect reflected light 23 from the platelet concentrate 15 in the PC bag 10 during the measurement interval. The light detector 31 is also configured to generate a real-time reflectance signal representative of light reflected from the platelet concentrate 10 during the measurement interval. This reflectance signal is processed by a controller 40 connected to the detector system 30. The controller 40 thereby determines platelet swirling for the platelet concentrate 15 in the PC bag 10 based on the real-time reflectance signal and determines the quality parameter for the platelet concentrate 15 in the PC bag 10 based on the platelet swirling.

In the embodiment shown in FIGS. 1A and 1B, the light system 20 and the detector system 30 thereby comprise one light source 21 and one light detector 31, respectively. The light source 21 and the light detector 31 are arranged on a same side relative to the PC bag 10 and the movable bag holder. Furthermore, the light source 21 is arranged on this side to direct light 22 into the platelet concentrate 15 in the PC bag 10 at an angle of incidence α that is selected so that the light detector 31 can detect reflected light 23 from the platelet concentrate 15 in the PC bag 10. Accordingly, this angle of incidence α is selected within an interval of from 5° to 85°. In preferred embodiment, the angle of incidence α is selected within an interval of from 15° to 85° and preferably from 25° to 85°. In a particular embodiment, this angle of incidence α is at least 25°, preferably at least 30°, more preferably at least 35°, such as at least 40° or at least 45° but no more than 85°, preferably no more than 80°, more preferably no more than 75°. Hence, a preferred interval of the angle of incidence α is from 45° to 75°.

Experimental data as provided herein shows that the real-time reflectance signal is a good representative of platelet swirling caused by light diffraction due to the alignment of normal discoid-shaped platelets. These discoids align light that is diffracted, creating a cloud- or swirl-like appearance that is well captured by monitoring the real-time reflectance signal and in particular the reflectance signal amplitude. FIG. 9 illustrates real-time reflectance signal measured 2 days and 12 days after production of a platelet concentrate in a PC bag. As is shown in the figure, the reflectance signal variability or variance is significantly higher after 2 days when the quality of the platelets in the platelet concentrate is good, whereas the reflectance signal variability or variance is much smaller after 12 days when many of the platelets have died. Hence, the reflectance signal variability can advantageously be used to determine the quality parameter. This reflectance signal variability is higher for viable platelets (high or good quality) as represented after 2 days as compared to deteriorated platelets (low or poor quality) as represented after 12 days.

In an embodiment, the at least one light source 21 is configured to direct light 22 into the platelet concentrate 15 in the PC bag 10 at the angle of incidence α for a measurement interval of at least 0.5 s, preferably at least 1 s. In an embodiment, the measurement interval is no longer than 40 s, preferably no longer than 30 s, and more preferably no longer than 20 s. Hence, in an embodiment, the measurement interval is selected within a range of from 0.5 s up to 40 s, preferably within a range of from 1 s up to 30 s and more preferably within a range of from 1 s up to 20 s, such as 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s or 20 s, preferably from 1 s up to 10 s, or from 1 s up to 5 s. In this embodiment, the at least one detector 31 is configured to detect reflected light 23 from the platelet concentrate 15 in the PC bag 10 during the measurement interval and generate the real-time reflectance signal representative of light reflected from the platelet concentrate 15 during the measurement interval. The controller 40 is then configured to determine the platelet swirling for the platelet concentrate 15 in the PC bag 10 based on the real-time reflectance signal.

As mentioned in the foregoing, the controller 40 may, in an embodiment, determine reflectance signal average based on the real-time reflectance signal. In such an embodiment, the controller 40 is configured to determine the quality parameter based on the reflectance signal average and the real-time reflectance signal. For instance, the controller 40 may be configured to determine, for each signal sample of the real-time reflection signal, a difference between the sample value and the reflectance signal average and then determine the platelet swirling based on the calculated differences.

In an embodiment, the controller 40 is connected to the light system 20 and the detector system 20 and is configured to control a light source 21 of the light system 20 to direct light 22 into the platelet concentrate 15 in the PC bag 10 carried by the bag holder 2 at the angle of incidence α. The controller 40 is also configured to control a light detector 31 of the detector system 30 to detect reflected light 31 from the platelet concentrate 15 in the PC bag 10 and generate the real-time reflectance signal. Thus, in this embodiment, the light measurements are conducted at, preferably scheduled, measurement intervals. At such a measurement interval, the controller 40 activates the light source 21 to direct light 22 into the platelet concentrate 15 and preferably activates the light detector 31 to detect reflected light 31 from the platelet concentrate 15. Hence, the light source 21 and light detector 31 thereby only need to be active at the measurement interval and can otherwise be in an inactive or low power mode. The switch between inactive or low power mode to active or high power mode is then performed by the controller 40.

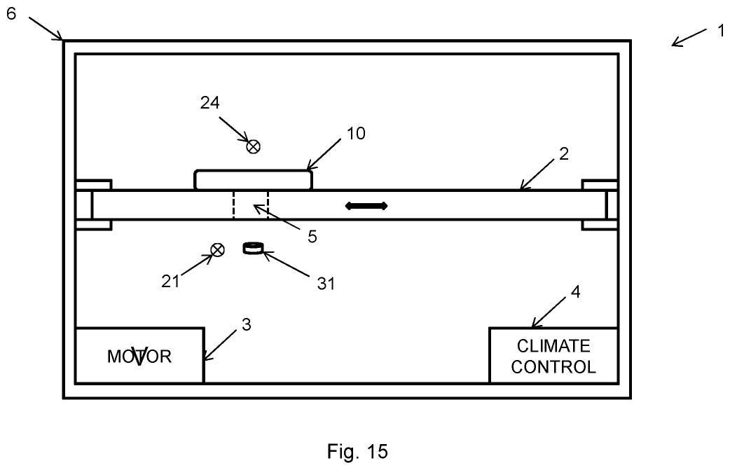

In an embodiment, the measurements are conducted at least once every X^{th} day for a selected value of X, such as 1 to 7, preferably 1 to 5, and more preferably 1 to 3. Hence, in an embodiment, the measurements are performed multiple times a week, such as at least twice a week, preferably at least three times a week. In an embodiment, the measurements are conducted at least once a day, or at least multiple times a day. In such a case, the measurements can be performed once every Y^{th} hour for a selected value of Y, such as 1 to 24, for instance 1 to 12, or 1 to 4. The manual visual inspection of platelet concentrates is typically performed once a day. Hence, in an embodiment, the measurements with the apparatus 1 of the present invention are also performed once a day. However, it may be advantageous to conduct the measurements more often than once a day, such as to filter out noise or artefacts. It is in fact possible to perform more or less continuous measurements according to the invention, in particular for an apparatus 1 providing horizontal movement of the PC bags 10 as shown in FIG. 15 and often referred to as Helmer-style.

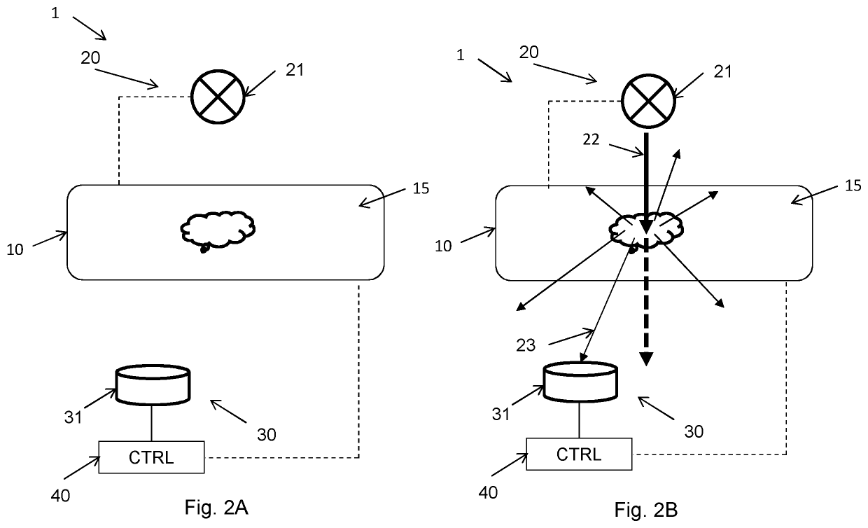

The apparatus 1 illustrated in FIGS. 1A and 1B is particularly adapted for real-time reflectance or reflection signal measurements. FIGS. 2A and 2B illustrate another embodiment of the apparatus 1 also adapted for real-time reflectance signal measurements. In this embodiment, the light system 20 also comprises a light source 21 and the detector system 30 comprises a light detector 31. However, in this embodiment, the light source 21 is arranged at a first side relative to the movable bag holder, whereas the light detector 31 is arranged at a second, opposite side relative to the movable bag holder.

As is schematically shown in the figures, the light detector 31 is preferably arranged relative to the light source 21 and the movable bag holder to be able to detect reflected light 23 from the platelet concentrate 15 in the PC bag 10 during the measurement interval. This means that the light detector 31 is preferably displaced relative to a light path corresponding to the incident light 22 and transmitted light (represented by bold hatched arrow in FIG. 2B) passing straight through the platelet concentrate 15 in the PC bag 10. Hence, the light detector 31 is preferably configured to detect light 23 exiting the PC bag 10 at a non-zero angle relative to the above mentioned light path.

As compared to the embodiment shown in FIGS. 1A and 1B, the embodiment shown in FIGS. 2A and 2B will detect reflected light in another direction relative to the platelet concentrate 15 and the PC bag 10.

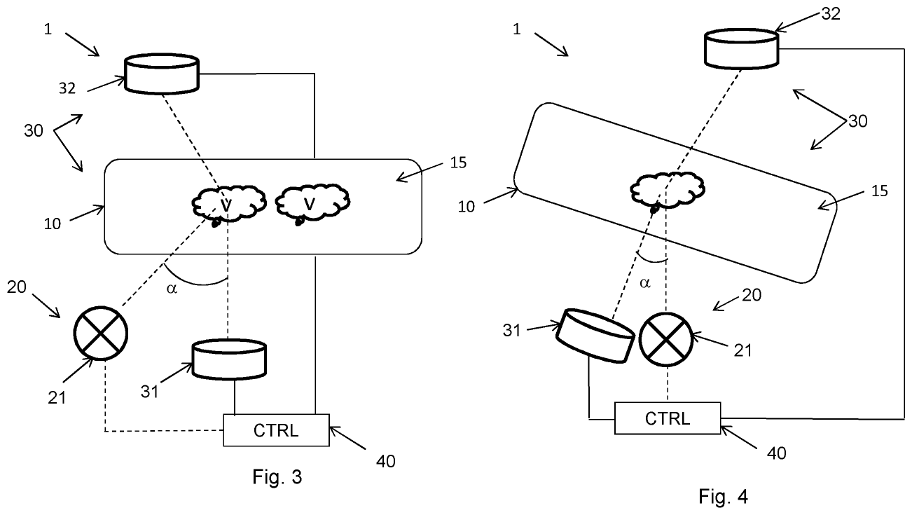

FIG. 3 illustrates yet another embodiment of the apparatus 1 configured to generate a real-time reflectance signal. In this embodiment, the light system 20 comprises a light source 21 but the detector system 30 comprises a first light detector 31 and a second light detector 32. The light source 21 and the light detector 31 are arranged at a first side relative to the movable bag holder, whereas the second light detector 32 is arranged at a second, opposite side relative to the movable bag holder. The first and second light detectors 31, 32 are then arranged to detect reflected light from the platelet concentrate 15 in the PC bag 10 during the measurement interval and generate a respective real-time reflectance signal representative of light reflected form the platelet concentrate 10 during the measurement interval. The two light detectors 31, 32 are thereby arranged to detect reflected light from different angles or directions relative to the platelet concentrate 15 in the PC bag 10.

In this embodiment, the controller 40 is configured to determine the platelet swirling based on the first real-time reflectance signal from the first light detector 31 and the second real-time reflectance signal from the second light detector 32. For instance, the controller 40 could process each of the first and second real-time reflectance signal separately as discussed in the foregoing to, for instance, calculate a first sum of calculated differences between sample values in the first real-time reflectance signal and an average of the first real-time reflectance signal and a second sum of calculated differences between sample values in the second real-time reflectance signal and an average of the second real-time reflectance signal. The controller 40 may then determine platelet swirling based on the first and second sums. For instance, the controller 40 could determine platelet swirling based on an average of the first and second sums or a sum of the first and second sums.

In another embodiment, the controller 40 may instead determine an average real-time reflectance signal based on the first and second real-time reflectance signals and then determine the platelet swirling based on this average real-time reflectance signal, such as discussed in the foregoing.

FIG. 4 is an illustration of an apparatus 1 according to a further embodiment that is capable of performing real-time reflectance measurements. In this embodiment, the light system 20 comprises a light source 21 arranged at a first side relative to the movable bag holder and PC bag 10, whereas the detector system 30 comprises a first light detector 31 arranged at the first side relative to the movable bag holder and PC bag 10 and a second detector 32 arranged at a second, opposite side relative to the movable bag holder and PC bag 10.

In the embodiment as shown in FIG. 4, the PC bag 10 is preferably arranged on the movable bag holder (not shown in FIG. 4) to be angled with the angle α relative to a horizontal axis. This further means that some of the light will pass directly through the platelet concentrate 15 in the angled PC bag 10 and some of the light will be reflected from the platelet concentrate 15 in the angled PC bag 10 and be detected by the first light detector 31 and by the second light detector 32. The tilting of the PC bag 10 implies that light from the light source 21 is directed into the platelet concentrate 15 at the angle of incidence α.

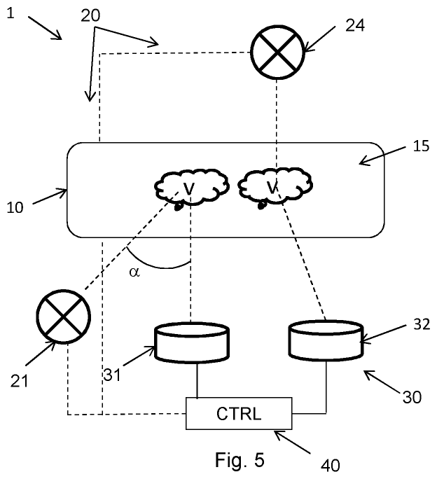

FIG. 5 illustrates yet another embodiment of the apparatus 1 that is basically a combination of the embodiment shown in FIGS. 1A and 1B and the embodiment shown in FIGS. 2A and 2B. In this embodiment, the apparatus 1 comprises a light system 20 comprising a first light source 21 and a second light source 24 configured to direct light into the platelet concentrate 15 in the PC bag carried by the movable bag holder 2 for a measurement interval. The detector system 30 of the apparatus 1 comprises a first light detector 31 and a second light detector 32 configured to detect reflected light from the platelet concentrate 15 in the PC bag 10 during the measurement interval.

In this embodiment, the first light source 21 and the first and second light detectors 31, 32 are arranged at a first side relative to the movable bag holder with the second light source 24 arranged at a second, opposite side relative to the movable bag holder.

The first light detector 31 is then arranged to detect reflected light originating from the first light source 21, whereas the second light detector 32 is arranged to detect reflected light originating from the second light source 24.

The controller 40 could be configured to activate both the first and second light sources 21, 24 at the same time so that the first and second light detectors 31, 32 detect reflected light during the same measurement interval. Alternatively, the controller 40 could sequentially activate the first and second light sources 21, 24 and the first and second light detectors 31, 32 to alternate between detection of reflected light by the first light detector 31 during a first measurement interval and detection of reflected light by the second light detector 32 during a second measurement interval. The first and second measurement intervals are preferably of the same duration but could alternatively be of different durations. As in the embodiments shown in FIGS. 3 and 4, first and second real-time reflectance signals are generated by the first and second light detectors 31, 32, respectively.

In another embodiment, the real-time detection signal is a real-time transmittance signal, i.e., representative of light having passed through the platelet concentrate in the PC bag during the measurement interval. In such an embodiment, the detector system comprises a light detector configured to detect light having passed through the platelet concentrate in the PC bag during the measurement interval and generate a real-time transmittance signal representative of light transmitted through the platelet concentrate during the measurement interval. In this embodiment, the controller is configured to determine the platelet swirling based on the real-time transmittance signal.

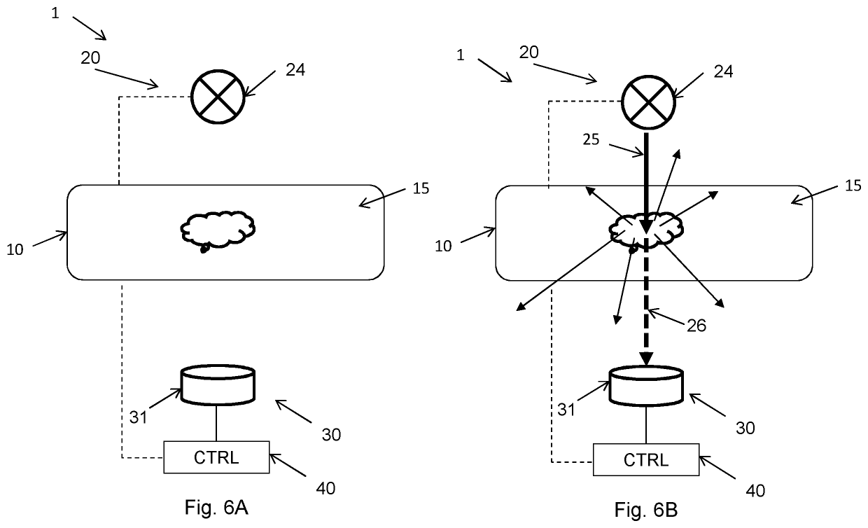

FIGS. 6A and 6B illustrate an embodiment of the apparatus 1 adapted for real-time transmittance signal measurements. In this embodiment, the light system 20 of the apparatus 1 comprises a light source 24 configured to direct light 25 through the platelet concentrate 15 in the PC bag 10 carried by the movable bag holder for the measurement interval. The detector system 30 of the apparatus 1 comprises, in this embodiment, a light detector 31 configured to detect light 26 having passed through the platelet concentrate 15 in the PC bag 10 during the measurement interval and generate a real-time transmittance signal representative of light transmitted through the platelet concentrate 15 during the measurement interval.

In this embodiment, the controller 40 is configured to determine platelet swirling for the platelet concentrate 15 in the PC bag 10 based on the real-time transmittance signal and determine the quality parameter for the platelet concentrate 15 in the PC bag 10 based on the platelet swirling.

In the embodiment shown in FIGS. 6A and 6B, the light system 20 and the detector system 30 thereby comprise one light source 24 and one light detector 31, respectively. The light detector 31 is arranged at a first side relative to the movable bag holder and the light source 24 is arranged at a second, opposite side relative to the movable bag holder.

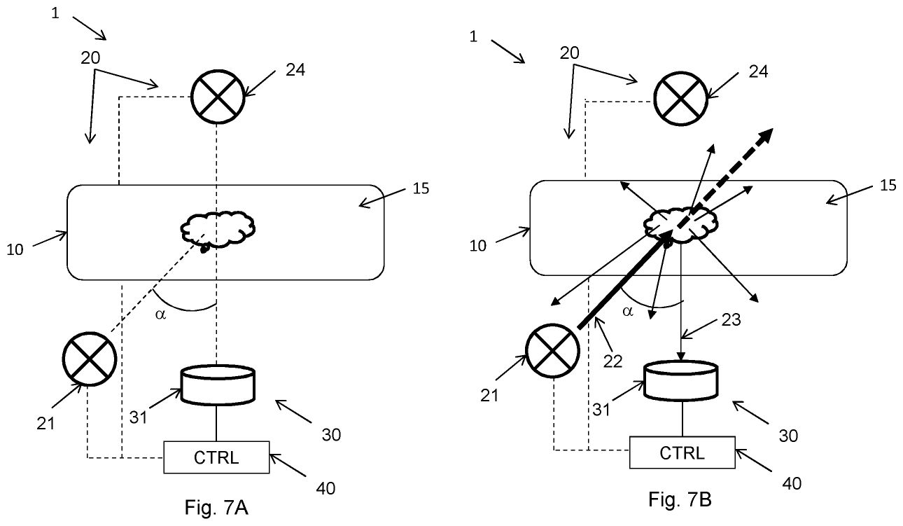

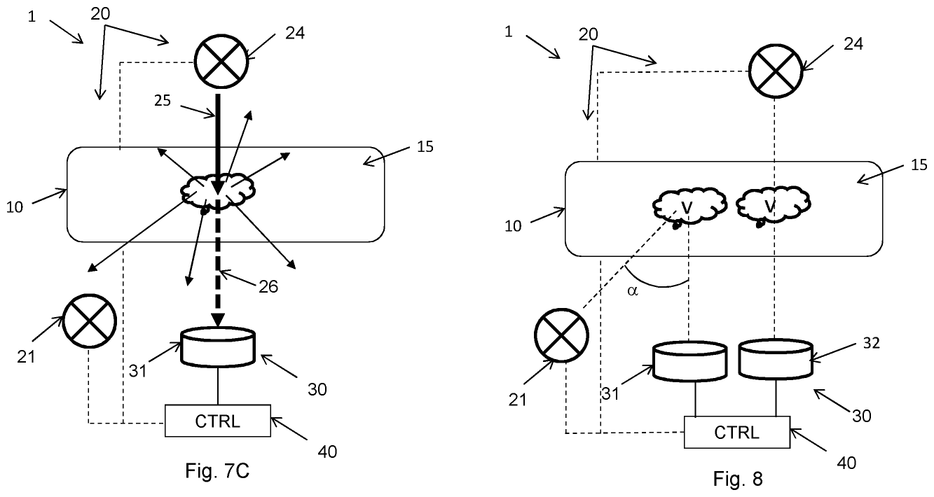

FIGS. 7A to 7C illustrate another embodiment of the apparatus 1 that comprises a light system 20 with a first light source 21 arranged at a first side relative to the movable bag holder and the PC bag 10 and a second light source 24 arranged at a second, opposite side relative to the movable bag holder and the PC bag 10. In this embodiment, the detector system 30 comprises a light detector 31 arranged at the first side relative to the movable bag holder and PC bag 10. The first light source 21 is then configured to direct light 22 into the platelet concentrate 15 in the PC bag 10 carried by the movable bag holder at the angle of incidence α, see FIG. 7B. Correspondingly, the second light source 24 is configured to direct light 25 through the platelet concentrate 15 in the PC bag 10 carried by the movable bag holder, see FIG. 7C.

Thus, in this embodiment as shown in FIGS. 7A to 7C, the apparatus 1 has a light system 20 comprising at least one light source 21 used for real-time reflectance measurements and at least one light source 24 used for real-time transmittance measurements. The respective light sources 21, 24 are then arranged on either side of the PC bag 10 with the at least one light source 21 for real-time reflectance measurements on the same side relative to the PC bag 10 as the detector 31.

In an embodiment, the controller 40 is connected to the light system 20 and the detector system 30. The controller 40 is configured to control a light source 24 of the light system 20 to direct light 25 through the platelet concentrate 15 in the PC bag 10 carried by the movable bag holder for a first measurement interval and control a light detector 31 of the detector system 30 to detect light 26 having passed through the platelet concentrate 15 in the PC bag 10 for the first measurement interval and generate the real-time transmittance signal. The controller 40 is also configured to control a light source 21 of the light system 20 to direct light 22 into the platelet concentrate 15 in the PC bag 10 carried by the movable bag holder at the angle of incidence α for a second measurement interval and control a light detector 31 of the detector system 30 to detect reflected light 23 from the platelet concentrate 15 in the PC bag 10 for the second measurement interval and generate the real-time reflectance signal.

The real-time reflectance measurements and the real-time transmittance measurements could be scheduled according to various embodiments. For instance, the apparatus 1 in FIGS. 7A to 7C could alternative between measuring reflectance and transmittance or could measure one of reflectance and transmittance more often than the other. In above, real-time transmittance measurements are conducted during a first measurement interval, whereas the real-time reflectance measurements are conducted during a second measurement interval. The first measurement interval may precede the second measurement interval or may follow the second measurement interval. Hence, the real-time transmittance measurements may, in the embodiment shown in FIGS. 7A to 7C, be performed prior to or after the real-time reflectance measurements.

In an embodiment, the controller 40 controls the light sources 21, 24 and preferably the detector 31 to perform real-time reflectance and/or transmittance measurements at scheduled measurement intervals, i.e., the first and second measurement intervals. Hence, in an embodiment, the controller 40 is configured to control the first light source 21 to direct light 22 into the platelet concentrate 15 in the PC bag 10 carried by the movable bag holder at the angle of incidence α (FIG. 7B) and control the second light source 24 to direct light 25 through the platelet concentrate 15 in the PC bag 10 carried by the movable bag holder (FIG. 7C). In this embodiment, the light system 20 comprises the first light source 21 arranged at a first side relative to the movable bag holder and the PC bag 10 and the second light source 24 arranged at a second, opposite side relative to the movable bag holder and the PC bag 10. The detector system 30 comprises a light detector 31 arranged at the first side relative to the movable bag holder and the PC bag 10. In such a case, the controller 40 is preferably also configured to control the light detector 31 to detect reflected light 23 from the platelet concentrate in the PC bag 10 and generate the real-time reflectance signal and control the light detector 31 to detect light 26 having passed through the platelet concentrate 15 in the PC bag 10 and generate the real-time transmittance signal.

In an embodiment, the controller 40 is configured to alternately control the first light source 21 to direct light 22 into the platelet concentrate 15 in the PC bag 10 carried by the movable bag holder at the angle of incidence α and control the second light source 24 to direct light 25 through the platelet concentrate 15 in the PC bag 10 carried by the movable bag holder. Hence, in this particular embodiment, the apparatus 1 alternate between conducting real-time reflectance measurements and real-time transmittance measurements.

In an embodiment, the controller 40 is configured to determine a transmittance signal average based on the real-time transmittance signal and determine the quality parameter based on the platelet swirling and the transmittance signal average.

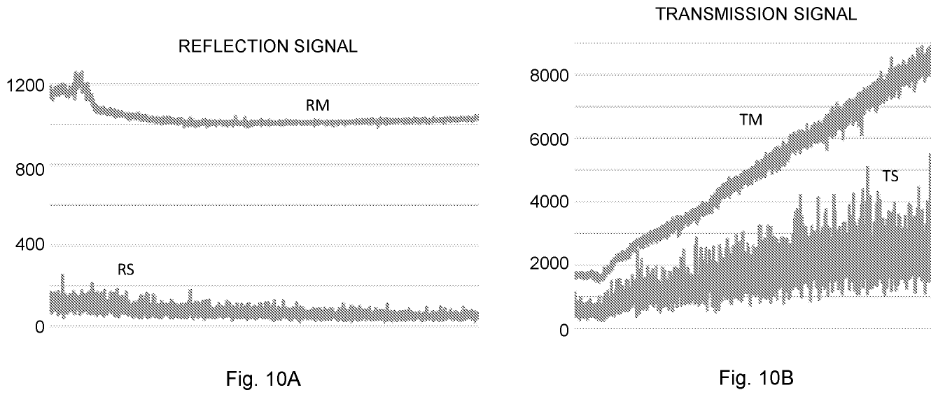

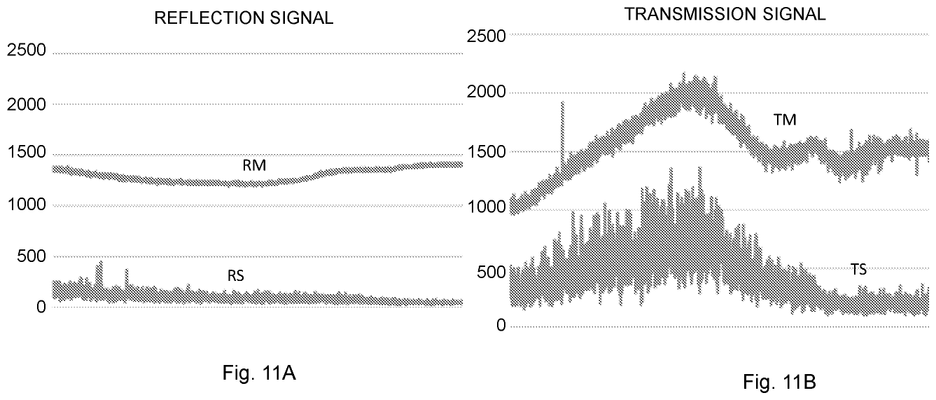

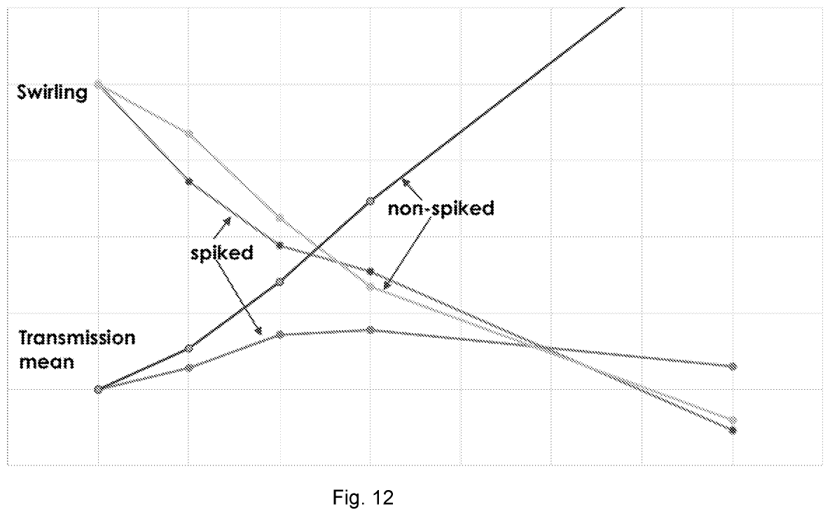

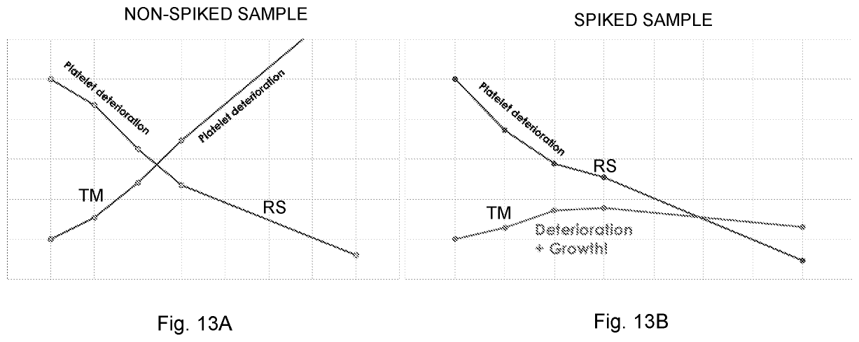

Experimental data as presented herein shows that real-time detection signal, i.e., the real-time reflection or transmittance signal, represents platelet quality better as compared to transmittance signal average. However, the transmittance signal average could be used in combination with the real-time detection signal to detect contamination in the platelet concentrate as shown by comparing FIGS. 10B and 11B, see also FIG. 12 and FIGS. 13A and 13B. Thus, the real-time detection signal accurately represents platelet swirling and platelet quality and changes as platelets deteriorate and die. The real-time detection signal furthermore accurately reflects the platelet quality independently of any microorganism contamination as shown by comparing FIGS. 10A and 11A, see also FIG. 12 and FIGS. 13A and 13B. If a change in real-time detection signal, see FIGS. 12 and 13A, is accompanied by a change in transmittance signal average, see FIGS. 12 and 13A, then this indicates a decline in platelet quality without any contamination. However, if the change in real-time detection signal is accompanied with substantially no change in transmittance signal average, then this is an indication of deterioration in platelet quality and contamination of the platelet concentrate 15, see FIGS. 12 and 13B.

Hence, in an embodiment, the controller 40 is configured to determine platelet viability based on the real-time detection signal and determine any cell contamination based on the transmittance signal average.

FIG. 8 is an illustration of an apparatus 1 according to another embodiment that is capable of performing both real-time reflectance and transmittance measurements. In this embodiment, the light system 20 comprises a first light source 21 arranged at a first side relative to the movable bag holder and PC bag 10 and a second light source 24 arranged at a second, opposite side relative to the movable bag holder and PC bag 10 as described above in connection with FIGS. 7A to 7C. The detector system 30 comprises a first detector 31 and a second detector 32 arranged at the first side relative to the movable bag holder and PC bag 10. In this embodiment, the first light source 21 is configured to direct light 22 into the platelet concentrate 15 in the PC bag 10 carried by the movable bag holder at the angle of incidence α. The first light detector 31 is then configured to detect reflected light 23 from the platelet concentrate 15 in the PC bag 10 and generate the real-time reflectance signal. The second light source 24 is configured to direct light 25 through the platelet concentrate 15 in the PC bag 10 carried by the movable bag holder and the second light detector 32 is configured to detect light 26 having passed through the platelet concentrate 15 in the PC bag 10 and generate the real-time transmittance signal.

Hence, the embodiment as shown in FIG. 8 has at least one pair of light source 21 and light detector 31 for real-time reflectance measurement and at least one pair of light source 24 and light detector 32 for real-time transmittance measurement. The embodiment as shown in FIG. 8 could be configured to perform the real-time transmittance and reflectance measurements sequentially as discussed above for the embodiment as shown in FIGS. 7A to 7C or at least partly in parallel, i.e., at least partly simultaneously.

In an aspect of the invention, the apparatus could be capable of performing real-time transmittance measurements, either alone or in combination with real-time reflectance measurements. In such an aspect, the apparatus 1 for determining quality of a platelet concentrate 15 in a PC bag 10 comprises a movable bag holder configured to carry the PC bag 10. The apparatus 1 also comprises a light system 20 comprising at least one light source 24 configured to direct light 25 through the platelet concentrate 15 in the PC bag 10 carried by the movable bag holder during a measurement interval. The apparatus 1 further comprises a detector system 30 comprising at least one light detector 31, 32 configured to detect light 26 having passed through the platelet concentrate 15 in the PC bag 10 during the measurement interval and generate a real-time transmittance signal representative of, preferably real-time, light transmitted through the platelet concentrate 15. The apparatus 1 also comprises a controller 40 connected to the detector system 30 and configured to determine platelet swirling for the platelet concentrate 15 in the PC bag 10 based on the real-time transmittance signal and determine a quality parameter for the platelet concentrate 15 in the PC bag 10 based on the platelet swirling.

In this aspect, the real-time transmittance signal is representative of real-time time light transmitted through the platelet concentrate 15 during the measurement signal. The real-time transmittance signal is thereby representative of platelet swirling for the platelet concentrate 15 in the PC bag 10.

The real-time transmittance signal may be processed by the controller as discussed in the foregoing, such as calculating an average of sample values and calculating differences between individual sample values and the calculated average.

The measurement interval is least 0.5 s, preferably at least 1 s. In an embodiment, the measurement interval is no longer than 40 s, preferably no longer than 30 s, and more preferably no longer than 20 s. Hence, in an embodiment, the measurement interval is selected within a range of from 1 s up to 20 s, such as 1 s, 2 s, 3 s, 4 s, 5 s, 6 s, 7 s, 8 s, 9 s, 10 s, 11 s, 12 s, 13 s, 14 s, 15 s, 16 s, 17 s, 18 s, 19 s or 20 s, preferably from 1 s up to 10 s, or from 1 s up to 5 s.

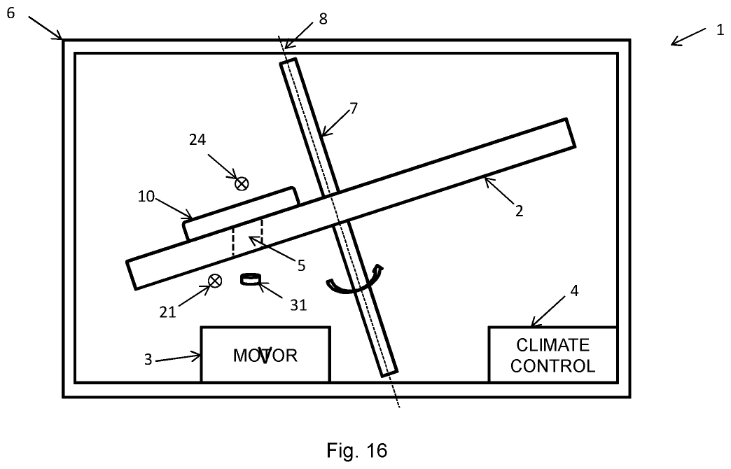

In an embodiment, the apparatus 1 comprises a motor 3 (see FIGS. 15 and 16) configured to move the movable bag holder 2, such as horizontally back and forth (FIG. 15), or by rotation around a rotation axis 8 (FIG. 16). In such an embodiment, the controller 40 is preferably configured to control the motor 3 to temporarily stop the movement of the movable bag holder 2 at the start of the measurement interval, or at least closely preceding the start of the measurement interval. The controller 40 is also configured to activate the at least one light source 24 and control the at least one light source 24 to direct light 25 through the platelet concentrate 15 in the PC bag 10. In an embodiment, the controller 40 may also be configured to activate the at least one detector 31, 32 and control the at least one detector 31, 32 to detect light 26 having passed through the platelet concentrate 15 in the PC bag 10 during the measurement interval.

The temporary stop of the movement of the movable bag holder 2 and thereby of the platelet concentrate 15 in the PC bag 10 carried by the movement bag holder 2 causes a movement of the platelets in the platelet concentrate 15, which is detected as a swirling effect by detection of the light 26 having passed through the platelet concentrate 10. This movement of the platelets in the platelet concentrate 15 is typically in the form of a non-laminar flow or movement of the platelets inside the PC bag 10. This (non-laminar) movement following the stop of the movement, and thereby the swirling effect, is dependent on the quality of the platelets due to a change in the form of the platelets from discoid-shaped for viable platelets to round-shaped for dead platelets. This means that the real-time detection signal generated by the at least one detector 31, 32 is dependent on the swirling effects of the platelets in the platelet concentrate 15.

A real-time reflection or transmittance signal representing detected reflected light 23 or light 26 having passed through the platelet concentrate 15 for an extended period of time, i.e., during the measurement interval, more accurately reflects the platelet swirling as compared to a single, instant measurement or detection of transmitted light 26. Furthermore, detecting reflected light 23 or transmitted light 26 reflected from or having passed through the platelet concentrate 15 immediately, or at least in connection with, stopping the movement of the platelet concentrate 15 and the PC bag 10 gives a more accurate representation of the swirling effect as compared to a single detection of light 23, 26 as the platelet concentrate 15 passes the at least one light source 24 and the at least one detector 31, 32, i.e., without stopping the movement of the movable bag holder 2.

FIGS. 15 and 16 illustrate two different embodiments of an apparatus 1 for determining quality of a platelet concentrate. In these two embodiments, a light system 20 and detector system 30 as shown in FIGS. 7A to 7C have been used as an illustrative example. In other embodiments, a light system 20 and detector system 30 as shown in FIGS. 1A and 1B, in FIGS. 2A and 2B, in FIG. 3, in FIG. 4, FIG. 5, in FIGS. 6A and 6B or FIG. 8 could be used.

The apparatus 1 comprises a cabinet or cupboard 6 enclosing one or multiple, i.e., at least two, movable bag holders 2, each carrying one or multiple PC bags 10. The cabinet 6 preferably has a door (not shown) that can be opened to enable placement of PC bags 10 on the movable bag holders 2 and retrieval of PC bags 10 therefrom. The door is generally closed to maintain a controlled internal environment within the cabinet 6. Such a climate control is preferably achieved using equipment 4 configured to maintain the controlled internal environment. Such equipment 4 may include temperature control equipment, such as heater and/or cooler, to maintain the temperature inside the cabinet 6 within a preferred interval, such as about 20-22° C. The equipment 4 may also include equipment for controlling the oxygen concentration (oxygen partial pressure), carbon dioxide concentration (carbon dioxide partial pressure) and/or water vapor content inside the cabinet 6.

The apparatus 1 shown in FIG. 15 comprises a movable bag holder 2 that is movable horizontally as indicated by the double-ended arrow. The apparatus 1 then comprises a motor 3 for horizontally moving the movable bag holder 2 back and forth. The motor 3 and movable bag holder 2 thereby provides an agitation of the platelet concentrate 15 in the PC bags 10. Hence, in an embodiment, the apparatus 1 provides continuous side-to-side motion, for instance using DELRIN® rollers and glides to allow movement and agitation of the PC bags 10 on the movable bag holders 2. Such agitation is used to prevent aggregation of the platelets and to optimize oxygenation of the platelets in the PC bags 10.

FIG. 16 illustrates another embodiment of the apparatus 1. In this embodiment, the at least one movable bag holder 2 is rotatable about a rotation axis 8 and forms an angle of inclination to a vertical axis. The apparatus 1 further comprises a motor 3 for rotating the movable bag holder 2 about the rotation axis 8 as indicated by the arrow. For instance, the apparatus 1 may contain a rotor 7 aligned with the rotation axis 8 and supporting one or more parallel planes or discs as movable bag holders 2. In an embodiment, the rotor 7 and rotation axis 8 are inclined so that the each PC bag 10 on a movable bag holder 2 moves between different vertical positions within the cabinet 6 during one full rotation, i.e., passes a higher point and a lowest point. In such an embodiment, the at least one light source 21, 24 and the at least one detector 31 are preferably arranged to in vicinity of a lowest point of the bag holder 2 as shown in FIG. 16.

The required agitation of the platelet concentrate in the PC bags 10 is achieved by the rotation of the movable bag holders 2 around the rotation axis 8 in FIG. 16.

The movable bag holders 2 may optionally comprise an opening or aperture 5 aligned with PC bags 10 as indicated in FIGS. 15 and 16. This aperture 5 then enables light from a light source 24 to pass through the platelet concentrate in the PC bag 10 and the movable bag holder 2 to reach the detector 31.

An advantage of the apparatus 1 as shown in FIG. 15 over the apparatus as shown in FIG. 16 is that light measurements can be performed without stopping the movement of the movable bag holder 2. In the apparatus 1 of FIG. 16, the rotation of the rotor 7 and the bag holders 2 must generally be stopped in order to be able to provide sufficient light measurement interval. However, if the rotation of the movable bag holders 2 is kept sufficient slow and the detection response of the at least one light detector 31 is sufficiently fast then light measurements can be conducted with the apparatus 1 in FIG. 16 even with a continuous rotation of the movable bag holders 2.

The at least one light source 21, 24 of the light system 20 could be any light source capable of providing light into and optionally through the platelet concentrate. In an embodiment, the at least one light source is at least one broadband light source 21, 24 configured to emit light in the visible spectrum, such as white light. In other embodiments, the light source may emit light within a selected range of the visible spectrum or indeed outside of the visible spectrum, such as in the infrared (IR) or near infrared (NIR) spectrum. It is also possible to use a light source or light sources configure to emit light within multiple ranges of the visible spectrum. Non-limiting examples of a light source 21, 24 of the apparatus 1 include light emitting diode (LED), such as a white LED, any colored LED, an IR LED or a NIR LED, and halogen light bulb.

The at least one light detector 31, 32 could be any detector that is capable of detecting reflected and/or transmitted light from the platelet concentrate 15. A non-limiting example of a light detector 31, 32 of the apparatus 1 is a photodiode. Other examples of light detectors 31, 32 include Avalange Photodiode (APD) and Silicon Photomultiplier (SiPM).

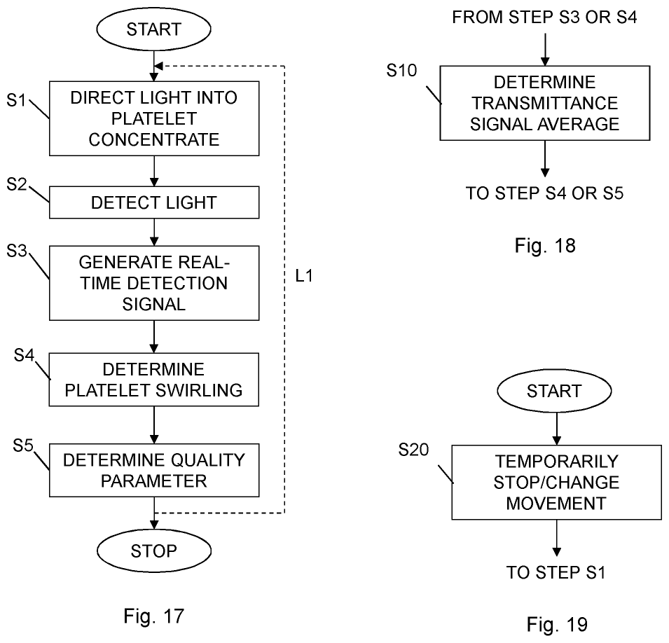

Another aspect of the embodiments relates to a method of determining quality of a platelet concentrate 15 in a PC bag 10, see FIG. 17. The method comprises directing, in step S1 and for or during a measurement interval, light 22, 25 into the platelet concentrate 15 in the PC bag 10 carried by a movable bag holder 2 configured to carry the PC bag 10 and agitate platelets in the PC bag 10. The method also comprises detecting, in step S2, light 23, 36 from the platelet concentrate 15 in the PC bag 10 and generating, in step S3, a real-time detection signal based on the detected light 23, 26. The method further comprises determining, in step S4, platelet swirling for the platelet concentrate 15 in the PC bag 10 based on the real-time detection signal and determining, in step S5, a quality parameter for the platelet concentrate 15 in the PC bag 10 based on the platelet swirling.

The method steps S1 to S5 are preferably performed at multiple times as indicated by the line L1 to determine and monitor the quality parameter for the platelet concentrate over time.

In an embodiment, the real-time detection signal comprises a plurality of signal samples having a respective sample value. In this embodiment, the method comprises calculating an average of the sample values of the signal samples. In this embodiment, step S4 comprises determining the platelet swirling based on the calculated and data points of the sample values of the signal samples.

In a particular embodiment, the method comprises calculating, for each signal sample of the real-time detection signal, a difference between the sample value of the signal sample and the calculated average. In this particular embodiment, step S4 comprises determining the platelet swirling based on the calculated differences, preferably based on a sum of the calculated differences.

In an embodiment, step S2 comprises detecting reflected light 23 from the platelet concentrate 15 in the PC bag 10 during the measurement interval and step S3 comprises generating a real-time reflectance signal representative of light reflected from the platelet concentrate 10 during the measurement interval.

In an embodiment, step S1 comprises directing light 22 into the platelet concentrate 15 in the PC bag 10 carried by the movable bag holder 2 at an angle of incidence α selected within an interval of from 5° to 85° for the measurement interval. In this embodiment, step S2 comprises detecting reflected light 23 from the platelet concentrate 15 in the PC bag 10 during the measurement interval and step S3 comprises generating a real-time reflectance signal representative of light reflected from the platelet concentrate 10 during the measurement interval.

In another embodiment, step S1 of FIG. 17 comprises directing light 25 through the platelet concentrate 15 in the PC bag 10 carried by the movable bag holder 2 for the measurement interval. Step S2 comprises, in this embodiment, detecting light 26 having passed through the platelet concentrate 15 in the PC bag 10 during the measurement interval. In this embodiment, step S3 comprises generating a real-time transmittance signal representative of light transmitted through the platelet concentrate 15 during the measurement interval.

In an embodiment, step S1 comprises directing light 25 through the platelet concentrate 15 in the PC bag 10 carried by the movable bag holder 2 for a first measurement interval and directing light 22 into the platelet concentrate 15 in the PC bag 10 carried by the movable bag holder 2 at an angle of incidence α selected within an interval of from 5° to 85° for the first measurement interval or a second measurement interval. Step S2 comprises, in this embodiment, detecting light 26 having passed through the platelet concentrate 15 in the PC bag 10 during the first measurement interval and detecting reflected light 23 from the platelet concentrate 15 in the PC bag 10 during the first or second measurement interval. Step S3 comprises generating a real-time transmittance signal representative of light transmitted through the platelet concentrate 15 during the first measurement interval and generating a real-time reflectance signal representative of light reflected from the platelet concentrate 10 during the first or second measurement interval. In this embodiment, step S4 comprises determining the platelet swirling for the platelet concentrate 15 in the PC bag 10 based on the real-time reflectance signal and/or of the real-time transmittance signal.

In this embodiment, a single same measurement interval, i.e., the first measurement interval, may be used for both reflectance and transmittance measurements. Alternatively, the measurement interval for the transmittance measurements could be regarded as a first measurement interval that may different from, e.g., having a different duration than and/or occurring at a different point in time, the measurement interval for the reflectance measurements, which could be regarded as a second measurement interval.

In an embodiment, the method comprises an additional step S10 as shown in FIG. 18. The method continues from step S3 or S4. Step S10 comprises determining a transmittance signal average based on the real-time transmittance signal. The method then continues to step S4 or S5 in FIG. 17. In this embodiment, step S5 comprises determining the quality parameter based on the platelet swirling and the transmittance signal average.

In an embodiment, step S5 comprises determining platelet viability based on the platelet swirling and determining any cell contamination based on the transmittance signal average.

FIG. 19 is a flow chart illustrating an additional, optional step of the method shown in FIG. 17. In an embodiment, the method comprises temporarily stopping, in step S20, movement of the movable bag holder 2 at a start of the measurement interval or at least closely preceding the start of the measurement interval. In another embodiment, this step S20 comprises temporarily changing movement of the movable bag holder 2 at a start of the measurement interval or at least closely preceding the start of the measurement interval. In either case, the method then continues to step S1 in FIG. 17.

### EXAMPLES

Materials and Methods

Sample Preparation

PC samples were prepared by pooling a sufficient number of compatible PC specimens in order to get a volume of ˜500 mL. This volume was split into two aliquots of −250 mL each, of which one was subsequently contaminated (“spiked”) with Staphylococcus epidermidis, a common skin bacterium. Both aliquots were filled into standard PC storage bags.

Measurement Apparatus

An apparatus of the present invention as shown in FIG. 15, denoted PlateGuard™ herein, was equipped with a measurement setup according to FIG. 7A and being able to measure transmission and reflection alternately. Real-time transmission and reflection data was recorded and used to calculate the average (mean) value of the signal to get transmission average (TM) (“turbidity”) and reflection average (RM), as well as the time-dependent detection signal (signal strength of the signal) (|sᵢ−s̅|), which represents a quantitative determination of swirling, i.e., to get transmission swirling (TS) and reflection swirling (RS).

Measuring Process

The PlateGuard™ device was loaded with the two prepared PC bags immediately after preparation. Immediately after preparation, as well as every following workday morning at the same time, a small sample (˜10 mL) was taken from each bag for laboratory analysis. In total, laboratory analysis was done on days 1, 2, 3, 4 and 8 (after expiry).

The PC bags were left for storage in the PlateGuard™ device for 8 days. During that time, the four parameters RM, RS, TM and TS were collected for both bags in a rapid sequence (with about one parameter per minute). The collected data was stored for analysis until after the 8 days of runtime.

Results

FIGS. 13A and 13B show the change of parameters TM and RS over time for the non-spiked and for the spiked sample, respectively. FIG. 12 shows the same as a combined graph.

FIG. 13A shows that the TM signal (“turbidity”) changes almost linearly over time, which was interpreted as platelet deterioration in WO 90/14588 and U.S. Pat. No. 6,288,778. However, the TM signal behaves drastically differently when turbidity of the platelet concentrate changes due to growing bacteria in the platelet concentrate (FIG. 13B). However, the RS signal (“swirling”) is not impacted by a change in turbidity and the RS signal is almost identical in both the spiked and the non-spiked platelet concentrate and thereby represents true platelet swirling.

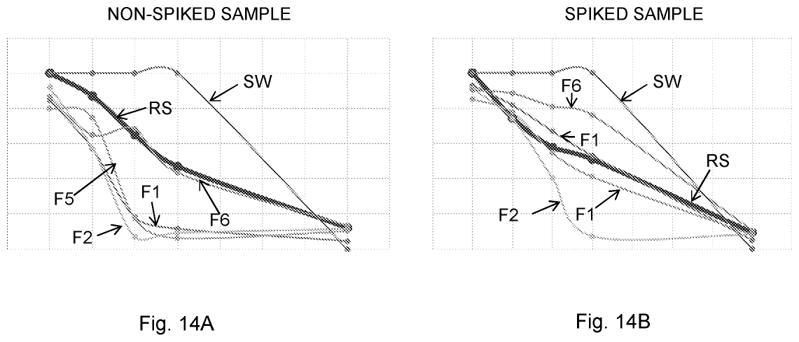

FIGS. 14A and 14B compare graphs of various interpretation methods for platelet viability, including visual inspection of the swirling effect by an experienced operator (ranged 0 to 3) (SW), quantitative determination of swirling by the PlateGuard™ device (RS), and various laboratory methods to determine platelet viability (F1-F6), aggregation at 16 μM collagen (F1), at 8 μM collagen (F2), at 0.5 mg/ml arachidonic acid (ARA) (F5) and at 20 μM thrombin receptor activating peptide (TRAP) (F6).

The RS signal shows an almost linear trend of the swirling effect while the eye of an experienced operator does not detect any change until after day 4. The laboratory data suggests that platelets have already deteriorated drastically at day 3, while the swirling effect is still good. However, RS according to the present invention shows a reduction in the quantitative determination of swirling down to 60% of the initial one.

### CONCLUSION

The present invention provides a quantitative determination of swirling, which in turn is a good representation of platelet viability. The monitoring of platelet quality according to the present invention can be performed even in the presence of bacterial growth, which otherwise will contaminate average transmission signal, i.e., turbidity, which therefore will not be representative of platelet viability in situations with microbial contamination.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-29. (canceled)

30. An apparatus for determining quality of a platelet concentrate (PC) in a PC bag, the apparatus comprising:
a movable bag holder configured to carry the PC bag and agitate platelets in the PC bag;
a light system comprising at least one light source configured to direct light into the platelet concentrate in the PC bag carried by the movable bag holder for a measurement interval;
a detector system comprising at least one light detector configured to detect light from the platelet concentrate in the PC bag during the measurement interval and generate a real-time detection signal; and
a controller connected to the detector system and configured to determine platelet swirling for the platelet concentrate in the PC bag based on the real-time detection signal and determine a quality parameter for the platelet concentrate in the PC bag based on the platelet swirling.

31. The apparatus according to claim 30, wherein
the real-time detection signal comprises a plurality of signal samples having a respective sample value; and
the controller is configured to
calculate an average of the sample values of the signal samples; and
determine the platelet swirling based on the calculated average and the sample values of the signal samples.

32. The apparatus according to claim 31, wherein the controller is configured to
calculate, for each signal sample of the real-time detection signal, a difference between the sample value of the signal sample and the calculated average; and
determine the platelet swirling based on the calculated differences.

33. The apparatus according to claim 32, wherein the controller is configured to determine the platelet swirling based on a sum of the calculated differences.

34. The apparatus according to claim 33, wherein the controller is configured to determine the platelet swirling based on Σᵢ₌₁^{N}|sᵢ−s̅=| or Σᵢ₌₁^{N}(sᵢ−s̅)², wherein the real-time detection signal comprises N signal samples for the measurement interval, sᵢ represents a sample value at sample number i, i=1 . . . N, and s̅ represents the calculated average.

35. The apparatus according to claim 30, wherein
the detector system comprises a light detector configured to detect reflected light from the platelet concentrate in the PC bag during, the measurement interval and generate a real-time reflectance signal representative of light reflected from the platelet concentrate during the measurement interval; and
the controller is configured to determine the platelet swirling based on the real-time reflectance signal.

36. The apparatus according to claim 35, wherein the light system comprises a light source configured to direct light into the platelet concentrate in the PC bag carried by the movable bag holder at an angle of incidence (a) selected within an interval of from 5° to 85° for the measurement interval.

37. The apparatus according to claim 36, wherein
the light source is arranged at a first side relative to the movable bag holder; and
the light detector is arranged at the first side relative to the movable bag holder.

38. The apparatus according to claim 36, wherein the light source is configured to direct light into the platelet concentrate in the PC bag carried by the movable bag holder at an angle of incidence (α) selected within an interval of from 15° to 85° for the measurement interval.

39. The apparatus according to claim 38, wherein the light source is configured to direct light into the platelet concentrate in the PC bag carried by the movable bag holder at an angle of incidence (α) selected within an interval of from 25° to 85° for the measurement interval.

40. The apparatus according to claim 39, wherein the light source is configured to direct light into the platelet concentrate in the PC bag carried by the movable bag holder at an angle of incidence (α) selected within an interval of from 45° to 75° for the measurement interval.

41. The apparatus according to claim 35, wherein
the light source is arranged at a first side relative to the movable bag holder; and
the light detector is arranged at a second, opposite side relative to the movable bag holder.

42. The apparatus according to claim 30, wherein
the light system comprises a light source configured to direct light through the platelet concentrate in the PC bag carried by the movable bag holder for the measurement interval;
the detector system comprises a light detector configured to detect light having passed through the platelet concentrate in the PC bag during the measurement interval and generate a real-time transmittance signal representative of light transmitted through the platelet concentrate during the measurement interval; and
the controller is configured to
determine a transmittance signal average based on the real-time transmittance signal; and
determine the quality parameter based on the platelet swirling and the transmittance signal average.

43. The apparatus according to claim 42, wherein the controller is configured to
determine platelet viability based on the platelet swirling; and
determine any cell contamination based on the transmittance signal average.

44. The apparatus according to claim 30, wherein the measurement interval has a duration is selected within a range of from 0.5 s up to 40 s.

45. The apparatus according to claim 44, wherein the measurement interval has a duration is selected within a range of from 1 s up to 30 s.

46. The apparatus according to claim 45, wherein the measurement interval has a duration is selected within a range of from 1 s up to 20 s.

47. The apparatus according to claim 30, wherein
the movable bag holder is movable horizontally; and
the apparatus further comprises a motor for horizontally moving the movable bag holder back and forth.

48. The apparatus according to claim 30, wherein
the movable bag holder is rotatable about a rotation axis; and
the apparatus further comprises a motor for rotating the movable bag holder about the rotation axis.

49. The apparatus according to claim 30, wherein the controller is configured to temporarily stop movement of the movable bag holder at a start of the measurement interval or at least closely preceding the start of the measurement interval.

50. The apparatus according to claim 30, wherein the controller is configured to temporarily change movement of the movable bag holder at a start of the measurement interval or at least closely preceding the start of the measurement interval.

51. The apparatus according to claim 30, wherein the at least one light source is configured emit light in the visible spectrum or within at least one selected range of the visible spectrum.

52. A method of determining quality of a platelet concentrate (PC) in a PC bag, the method comprises:
directing, for a measurement interval, light into the platelet concentrate in the PC bag carried by a movable bag holder configured to carry the PC bag and agitate platelets in the PC bag;
detecting light from the platelet concentrate in the PC bag during the measurement interval;
generating a real-time detection signal based on the detected light;
determining platelet swirling for the platelet concentrate in the PC bag based on the real-time detection signal; and
determining a quality parameter for the platelet concentrate in the PC bag based on the platelet swirling.

53. The method according to claim 52, wherein the real-time detection signal comprises a plurality of signal samples having a respective sample value, the method further comprising calculating an average of the sample values of the signal samples, wherein determining the platelet swirling comprises determining the platelet swirling based on the calculated and data points of the sample values of the signal samples.

54. The method according to claim 53, further comprising calculating, for each signal sample of the real-time detection signal, a difference between the sample value of the signal sample and the calculated average, and wherein determining the platelet swirling comprises determining the platelet swirling based on the calculated differences.

55. The method according to claim 54, wherein determining the platelet swirling comprises determining the platelet swirling based on a sum of the calculated differences.

56. The method according to claim 55, wherein determining the platelet swirling comprises determining the platelet swirling based on Σᵢ₌₁^{N}|sᵢ−s̅| or Σᵢ₌₁^{N}(sᵢ−s̅)², wherein the real-time detection signal comprises N signal samples for the measurement interval, sᵢ represents a sample value at sample number i, i=1 . . . N, and s̅ represents the calculated average.

57. The method according to claim 52, wherein
detecting light comprises detecting reflected light from the platelet concentrate in the PC bag during the measurement interval; and
generating the real-time detection signal comprises generating a real-time reflectance signal representative of light reflected from the platelet concentrate during the measurement interval.

58. The method according to claim 57, wherein directing light comprises directing light into the platelet concentrate in the PC bag carried by the movable bag holder at an angle of incidence (a) selected within an interval of from 5° to 85° for the measurement interval.

59. The method according to claim 52, wherein
directing light comprises directing light through the platelet concentrate in the PC bag carried by the movable bag holder for the measurement interval;
detecting light comprises detecting light having passed through the platelet concentrate in the PC bag during the measurement interval;
generating the real-time detection signal comprises generating a real-time transmittance signal representative of light transmitted through the platelet concentrate during the measurement interval, the method further comprises:
determining a transmittance signal average based on the real-time transmittance signal, wherein
determining the quality parameter comprises determining the quality parameter based on the platelet swirling and the transmittance signal average.

60. The method according to claim 59, wherein determining the quality parameter comprises determining platelet viability based on the platelet swirling and determining any cell contamination based on the transmittance signal average.

61. The method according to claim 52, further comprising temporarily stopping movement of the movable bag holder at a start of the measurement interval or at least closely preceding the start of the measurement interval.

62. The method according to claim 52, further comprising temporarily changing movement of the movable bag holder at a start of the measurement interval or at least closely preceding the start of the measurement interval.
